(19) United States
(12) Patent Application Publication
(10) Pub. No.: US 2009/0263479 A1
(43) Pub. Date: Oct. 22, 2009

(54) FORMULATIONS FOR POORLY PERMEABLE ACTIVE PHARMACEUTICAL INGREDIENTS

(75) Inventors: Jan P. Moschwitzer, Weesp (NL); Aldo V. Ket, Weesp (NL); Heike Dinter-Heidorn, Hannover (DE)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Solvay Phamaceuticals GmbH

(21) Appl. No.: 12/427,144

(22) Filed: Apr. 21, 2009

Related U.S. Application Data
(60) Provisional application No. 61/046,871, filed on Apr. 22, 2008.

(30) Foreign Application Priority Data
Apr. 22, 2008 (EP) 08103657.6

(51) Int. Cl. A61K 9/48; A61K 47/00; A61K 9/16

(52) U.S. Cl. 424/463; 514/772; 424/490

(57) ABSTRACT

The present invention relates to a pharmaceutical oral dosage form containing a poorly permeable active pharmaceutical ingredient and at least one permeability improving substance, wherein the permeability improving substance is thermostably embedded in a water-soluble matrix of a water soluble carrier, and to thermostable formulations which can be used to improve bioavailability.

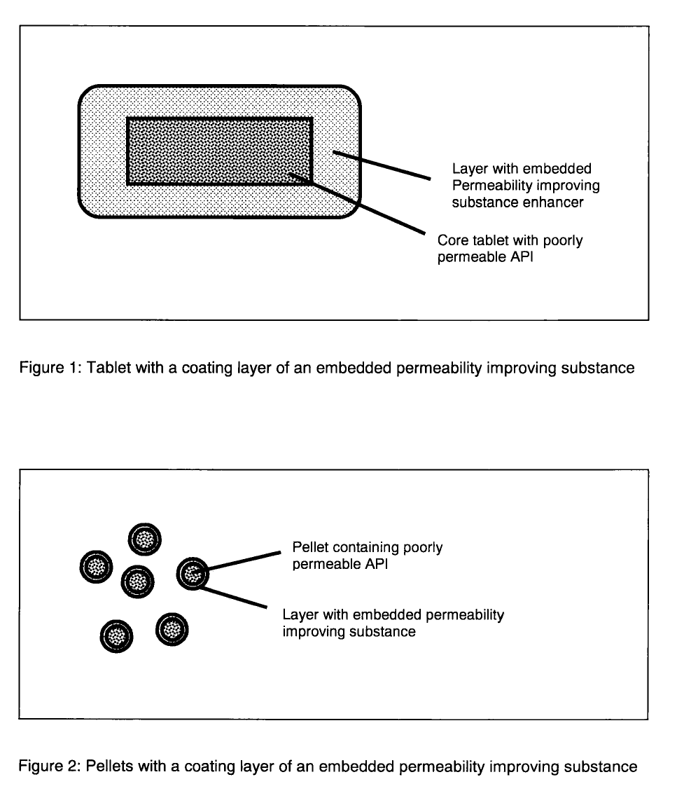

This application claims the benefit of U.S. provisional application No. 61/046,871, filed Apr. 22, 2008, the disclosure of which is incorporated herein by reference.

The present invention relates to formulations for poorly permeable active pharmaceutical ingredients, to thermostable solid formulations containing at least one permeability improving substance embedded in a water soluble carrier and to thermostable formulations which show an improved bioavailability or can be used to improve bioavailability.

The invention also relates to a pharmaceutical oral dosage form containing at least one poorly permeable active pharmaceutical ingredient and at least one permeability improving substance, which is thermostably embedded in a water-soluble matrix of a water soluble carrier, such as a pharmaceutically acceptable carrier by employing an atomization technique together with a drying step.

### BACKGROUND OF THE INVENTION

Many active pharmaceutical ingredients (APIs) show poor bioavailability after oral administration. Numerous examples are given in the literature of how oral bioavailability can be improved when APIs have poor aqueous solubility but good permeability. These compounds are referred to as BCS Class II compounds according to the biopharmaceutics classification system (BCS system (G. L. Amidon, H. Lennernas, V. P. Shah, and J. R. Crison. A theoretical basis for a biopharmaceutics drug classification: the correlation of in vitro drug product dissolution and in vivo bioavailability. Pharm. Res. 12:413-420 (1995)). In contrast to this, the literature describing techniques to improve the oral bioavailability of poorly permeable active pharmaceutical ingredients, often referred to as BCS Class III or BCS Class IV compounds, is relatively rare. The patent literature is replete with examples of permeation enhancers which effectively increase the parenteral permeability of drugs, e.g., in transdermal drug delivery systems. The examples for orally administered compounds are distinctly lower.

According to the biopharmaceutics classification system, APIs belonging to BCS Class III possess good aqueous solubility but poor permeability to biological membranes. Poorly permeable active pharmaceutical ingredients are often poorly absorbed through oral and other mucosa due to the limitations of their physicochemical properties. Some physicochemical properties that have been associated with poor membrane permeability are low octanol/aqueous partitioning (log P), the presence of strongly charged functional groups, high molecular weight, a substantial number of hydrogen-bonding functional groups, and high polar surface area. For some compounds, permeation through the intestinal epithelium is hindered by their active transport from the enterocyte back into the intestinal lumen. The secretory transporters involved may include P-glycoprotein (Pgp), (belonging to the ATP Binding Cassette (ABC) superfamily), the family of multidrug resistance-associated proteins (MRP), and possibly others. For substrates and modifiers of these secretory transporters, inhibiting secretory transport can increase permeation in the absorptive direction. (B. J. Aungst, J. of Pharm. Sci. 2000, 89(4), 429-44). The active pharmaceutical ingredients may benefit most from intestinal absorption-enhancing formulations.

In order to address the problem of poor permeability the literature suggests many permeability improving substances, like mucoadhesive polymers, pH modifiers, permeation enhancer and efflux inhibitors.

The term bioadhesion refers to any bond formed between two biological surfaces or a bond between a biological and a synthetic surface. In the instance in which bonds form between mucus (e.g. in the gastrointestinal tract) and polymer, the term mucoadhesion is used synonymously with bioadhesion (D. E. Chickering, E. Mathiowitz, Definitions, Mechanisms, and Theories of Bioadhesion. In: E. Mathiowitz, D. E. Chickering, C.-M. Lehr (Eds.) Bioadhesive drug delivery systems. Fundamentals, novel approaches, and development. Marcel Dekker Inc., New York). Mucoadhesive polymers are used to prolong the gastrointestinal residence time, and therefore lead to a better absorption of the poorly permeable active pharmaceutical ingredient.

Permeation enhancers increase the permeability of a mucosal barrier and facilitate the diffusion of an active pharmaceutical ingredient across the mucosal barrier by disrupting the mucosal barrier either by opening tight-junctions between adjacent epithelial cells (paracellular pathway) or by fluidizing phospholipid membranes to allow better diffusion of the active drug across the bilayer (transcellular pathway)(B. J. Aungst, J. of Pharm. Sci. 2000, 89(4), 429-44; J. Hochman et al., “Mechanisms of absorption enhancement and tight junction regulation”, J. Control. Rel. 29:253-267).

Efflux inhibitors are substances capable of enhancing the permeability of active pharmaceutical ingredients which are hindered by their active transport from the enterocyte back into the intestinal lumen via secretory transporters such as P-glycoprotein (Pgp), the family of multidrug resistance-associated proteins (MRP), and possibly others. Efflux inhibitors are substrates or modifiers of these secretory transporters, by inhibiting or modifying the secretory transport the permeation in the absorptive direction can be increased. In the framework of the present invention, efflux inhibitors are regarded as permeability improving substances.

US patent application publication 20050244502 describes a composition which enhances bioavailability of therapeutic agents which may be poorly absorbed, which composition contains a mucoadhesive and an absorption enhancer, and has surprisingly reduced toxicity as compared to previously known absorption enhancing compositions, a method for improving bioavailability of poorly absorbable therapeutic agents via oral or topical delivery to mucosal membranes employing such composition, and a method for reducing cytotoxic effects of an absorption enhancer (employed to improve bioavailability of poorly absorbed therapeutic agents) thereby providing more tolerable delivery to mucosal membranes, employing a special mucoadhesive in combination with the absorption enhancer. In order to obtain a solid formulation according to this invention, the mucoadhesive polymer and absorption enhancers are mixed with the remaining ingredients. For example, example 3 of this invention shows that high shear granulation/mixing was applied therefore. The composition is not referred to as thermostable.

U.S. Pat. No. 6,793,934 describes an immediate-release pharmaceutical composition comprising a liquid drug, drug solutions, and oral absorption enhancer solution or liquid oral absorption enhancers in the form of a free-flowing powder. The invention uses powdered solution technology, i.e., a carrier is used for turning a liquid agent into a dry, non-adherent, free-flowing compressible powder, when the administration of an active agent in a liquid formulation would be disadvantageous. A powder according to this invention can be considered free flowing if it meets the processing characteristics such that in the process of making tablets, the resulting tablet weights are uniform, or in the process of filling capsules, the resulting capsule weight is uniform. Drug-containing liquids are blended with either the granulated dibasic calcium phosphate or magnesium aluminometasilicate or in combination in a V-shaped blender to form a free-flowing, dry powder. The blending process also can be carried out in a planetary mixer, high shear granulator, fluid-bed granulator, or by a simple mixing using a spatter or other mixing methods known to one skilled in the art. Subsequently, the resulting powdered solution can be further blended with other pharmaceutical processing aids, such as bulking agent, disintegrant, glidant, and lubricant, then compressed into tablets on a rotary press using appropriate tooling. The formulation technique of this invention does not lead to a thermostable composition.

US patent application publication 2007292512 describes a pharmaceutical composition, particularly oral dosage forms, comprising a DAC inhibitor in combination with an enhancer to promote absorption of the DAC inhibitor at the GIT cell lining. The enhancer is a medium chain fatty acid or derivative thereof having a carbon chain length of from 6 to 20 carbon atoms. In certain embodiments, the solid oral dosage form is a controlled release dosage form, such as a delayed release dosage form. Blend or granulates containing permeation enhancers according to this invention were produced by a simple mixing step, either in a Kenwood Chef mixer or a high shear mixer (Gral 10). The formulation technique of this invention does not lead to a thermostable composition.

U.S. Pat. No. 6,692,771 describes novel emulsion compositions which improve the rate and/or extent of absorption of drugs. The emulsion compositions in this patent include drug-containing emulsions adsorbed onto solid particles which may be further formulated into solid dosage forms, methods of preparing such emulsion compositions and their uses thereof. The emulsion compositions and their dosage forms improve the drug-load and the bioavailability of a wide range of drugs, including drugs that are known or suspected of having poor bioavailability by the utilization of several different mechanisms. This invention again applies the powdered solution technology by simple adsorption of the emulsion composition onto solid particle adsorbent selected from the group consisting of kaolin, bentonite, hectorite, colloidal magnesium aluminum silicate, silicon dioxide, magnesium trisilicate, aluminum hydroxide, magnesium hydroxide, magnesium oxide and talc. The resulting compositions are therefore not thermostable.

International patent application publication WO 2008/046905 describes a thermostable solid composition comprising nanosized micelles, the micelles containing a poorly soluble chemical substance.

The prior art describes compositions where especially the liquid or semi-solid permeability improving substances are either simply mixed with other excipients to obtain a freely flowable powder, or the liquid or semi-solid permeability improving substances are adsorbed to a solid carrier. This requires normally high excipient amounts and leads either to a relatively low amount of permeability improving substances or a relatively large oral solid dosage form. The resulting solid dosage forms are also not thermostable, which means that e.g., liquid or semi-solid permeation enhancers or efflux inhibitors will leak out of the adsorbent when these solid dosage forms are exposed to elevated temperature where the liquid or semi solid permeation enhancers or efflux inhibitors exist in liquid state.

It is an objective of the present invention to provide further and improved formulations for water soluble but poorly permeable active pharmaceutical ingredients (BCS III compounds) that can be prepared by using commercially available materials and standard processes and equipment. These formulations are especially useful for active pharmaceutically ingredients with an even lower permeability than the normal BCSIII type compounds, which are defined below as compounds having a bad permeability. A further aim is to provide thermostable formulations which also show an improved bioavailability.

### DETAILED DESCRIPTION OF THE INVENTION

The present invention relates to compositions containing at least one permeability improving substance that can be used to improve the bioavailability of a poorly permeable active pharmaceutical ingredient and to pharmaceutical oral dosage forms containing a water soluble but poorly permeable active pharmaceutical ingredient and at least one permeability improving substance. The permeability improving substance is neither a mucoadhesive polymer nor a pH modifier. The permeability improving substance can be thermostably embedded in a water-soluble matrix of a water soluble carrier, such as a pharmaceutically acceptable carrier, by employing an atomization technique together with a drying step.

It is known, that it is difficult to incorporate liquid or semi-solid materials, such as surfactants or oils, for example, polysorbates (Tween® 20, 40, 60, 80), polyglycolized glycerides (Labrasol), and vegetable oil, etc., into a solid dosage form, especially in a tablet dosage form. In order to produce a conventional solid dosage form from a liquid poorly soluble drug the production of “powdered solutions” was suggested by Spireas et al. (Spireas et al., Powdered solution technology: principles and mechanisms, Pharm. Res. 1992, 9(10), 1351-1358). The “powdered solution” was produced by admixing the liquid drugs or drug solutions with a selected carrier. The product obtained by this technology is a physical mixture or blend of a drug/surfactant solution and the selected carrier. Examples of these kind of formulations are disclosed in WO 2005/041929, WO 2006/113631 and WO 2006/135480. However, a typical drawback of the resulting powder is its' poor flowability, especially its poor thermostability and poor compressability.

In the framework of the present invention it was surprisingly found that a permeability improving substance or mixture of permeability improving substances can be embedded into a water-soluble matrix of a water soluble carrier or a mixture of water soluble carriers by using atomization techniques together with drying techniques. According to the present invention the permeability improving substance is thermostably embedded in a matrix of a pharmaceutically acceptable carrier by using an atomization technique together with a drying technique, like spray-drying, spray-coating, spray-layering, spray-granulation of an aqueous solution of a pharmaceutically acceptable carrier together with an aqueous solution, or an aqueous micellar solution or an aqueous nanoemulsion, or an aqueous microemulsion or aqueous emulsion of the permeability improving substance. The above mentioned composition does not contain an active pharmaceutical ingredient, but can be used to improve the permeability of a poorly permeable active pharmaceutical ingredient.

The above mentioned composition according to the present invention comprises at least 10% of a permeability improving substance or at least 15%, at least 20%, at least 25%, at least 30%, at least 35%, at least 40%, at least 45%, at least 50% or at least 60% and may comprise up to 75% or 80% of a permeability improving substance.

The ratio between permeability improving substance and water soluble polymer can be 10:1, 8:1, 6:1, 5:1, 4:1, 2:1, 1:1 or 0.5:1 or 0.1:1, or all ratios between the indicated fixed ratios, such as between 10:1 and 8:1, between 8:1 and 6:1, between 6:1 and 5:1, between 5:1 and 4:1, between 4:1 and 2:1, between 2:1 and 1:1, between 1:1 and 0.5:1 and between 0.5:1 and 0.1:1, depending on the specific permeability improving substance and the specific water soluble polymer.

As used herein the term water soluble matrix means a matrix of a water soluble carrier or a mixture of water soluble carriers. The matrix forming material is defined as at least one water soluble carrier which is used to prepare the water soluble matrix.

The sum of the permeability improving substance and the water soluble matrix in the composition according to the present invention is at least 80% w/w, or at least 85% w/w, or at least 90% w/w, or at least 95% w/w, or at least 99% w/w of the total dry material in the composition. The term total dry material is the same as the term total dry substance as commonly used in the art.

In prior art formulations, compounds which can be used as permeability improving substances are often used as surfactants, as most permeability improving substances have also surfactant properties. In these prior art formulations said compounds are used in amounts considerably lower than the amounts used in the present invention as they are not aimed to improve the permeability of the active pharmaceutical ingredient, rather they are aimed to act as a wetting agent, solubility enhancing agent or plasticizer.

Permeability improving substances according to the present invention include, but are not limited to, the following: polyethylene glycol, propylene glycol, glycerin, vegetable oil, cotton seed oil, corn oil, peanut oil, sesame oil, mineral oil, glycofurol, propylene glycol dicaprylate/dicaprate, glyceryl caprylate/caprate, oleic acid, ethoxydiglycol, and poloxamer block copolymers, polysorbates, sorbitan esters, poloxamer block copolymers, PEG-35 castor oil, PEG-40 hydrogenated castor oil, caprylocaproyl macrogol-8 glycerides, sodium lauryl sulfate, dioctyl sulfosuccinate, polyethylene lauryl ether, ethoxydiglycol, propylene glycol monocaprylate, propylene glycol mono-di-caprylate, propylene glycol dicarpylate/dicparate, glyceryl monocaprylate, glyceryl mono-di-caprylate, caprylocaproyl macrogolglycerides (such as PEG caprylic/capric glycerides), glyceryl fatty acids (C₈-C₁₈) ethoxylated, oleic acid, linoleic acid, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1,000 succinate. In addition, a combination of oral absorption enhancers can be used to improve the absorption further.

Additional permeability improving substances according to the present invention are: d-alpha tocopheryl polyethylene glycol 1,000 succinate (Vit E TPGS), PEG-32 glyceryl laurate (e.g. Gelucire® 44/14), caprylic/capric acid triglyceride (e.g. Captex® 8000), glyceryl monocaprylate (e.g. Capmul® MCM C8), glyceryl mono-di-caprylate, polyethoxylated castor oil (e.g. Cremophor® EL), polyglycolyzed glycerides and polyoxyethylene esters of 12-hydroxystearic acid, medium chain triglycerides, caprylocaproyl macrogol-8 glycerides, polyoxyethylene-20 sorbitanmonooleate, macrogol-15 hydroxystearate, propylene glycol-monocaprylate (e.g. Capryol® 90 or Capryol® PGMC), propylene gycol-caprylcaprate (e.g. Labrafac® PG), and propylene glycol-monolaurate (e.g. Lauroglycol® 90 or Lauroglycol® FCC).

Also acceptable in the framework of the present invention are the specific permeability improving substances Labrasol®, Solutol® HS 15, Capmul® MCM C8, Captex® 8000, Vitamin E TPGS, Gelucire® 44/14, Cremophor® EL, Tween® 80, Miglyol® 812, Capryol® 90, Capryol® PGMC, Labrafac® PG, Lauroglycol® 90 and Lauroglycol® FCC.

In the framework of the present invention the term thermostable means that the composition remains a free flowing stable powder when heated above the melting point of the main permeability improving substance. If produced as a powder, the thermostable composition remains a free flowing stable powder when heated 5° C., 10° C., 15° C., 20° C., 25° C., 30° C. or 40° C. above the melting point of the main permeability improving substance. For example, Vitamin E TPGS (d-alpha-tocopheryl polyethylene glycol 1000 succinate) has a melting point of 36° C. (Reference: Eastman, Material Safety Data Sheet of Vit E TPGS NF Grade). A person skilled in the art would assume that if Vitamin E TPGS is the main component of a composition according to the present invention, this composition would show at least partial melting when exposed to a temperature far above 36° C., for instance 80° C. However, if the Vitamin E TPGS is used as a permeability improving substance for the present invention, that means that Vitamin E TPGS is embedded in a water-soluble matrix material with a melting point above 36° C., the resulting powder will not show a major change in powder morphology and flowability. It remains a stable, free-flowing powder even if exposed to temperatures 20 to 40° C. above the melting point of the permeability improving substance.

The thermostable composition comprising a permeability improving substance embedded in a water soluble carrier according to the present invention can be used to improve the bioavailability of a poorly permeable active pharmaceutical ingredient by mixing said thermostable composition with a powder, a granule, a pellet or microspheres comprising said poorly permeable active pharmaceutical ingredient or by applying a coating comprising said thermostable composition on a tablet core or a granule, a pellet or microspheres comprising said poorly permeable active pharmaceutical ingredient. The thermostable composition itself can be a powder, but may also be formulated into a granule, a pellet, a tablet or microspheres.

The present invention therefore also relates to a pharmaceutical composition comprising at least two different phases, wherein the first phase a) comprises an active pharmaceutical ingredient formulated into a powder, a granule, a pellet, a microsphere or a tablet; and the second phase b) comprises a thermostable solid composition as described above, and wherein said active pharmaceutical ingredient is a water soluble substance having a bad permeability.

In the framework of the present invention a water soluble substance means that at least one gram of the substance is soluble in 10 to 30 grams of water, or in 1 to 10 grams of water or in less than 1 gram of water (resp. soluble, freely soluble and very soluble according to the definition of the European Pharmacopeia 6.3)

According to the literature (Rautio et al., Nature Reviews Drug Discovery 2008, 7, 255-70) low or poorly permeable compounds (BCS class III) have a permeability when tested in Caco-2 cell lines of equal to or lower than 5×10⁻⁶ cm/sec. Therefore, in the framework of the present invention a poor permeability means a permeability when tested in Caco-2 cell lines according to Xin He et al. (Int. J. of Pharmaceutics 2003, 263, 35-44) of equal to or lower than 5×10⁻⁶ cm/sec.

In the framework of the present invention a bad permeability means a permeability when tested in Caco-2 cell lines according to Xin He et al. of equal to or lower than 0.5×10⁻⁶ cm/sec preferably equal to or lower than 0.2×10⁻⁶ cm/sec or equal to or lower than 1×10⁻⁷ cm/sec and an absolute oral bioavailability in humans lower than 20%, or even lower than 15%, and even lower than 10%, when formulated without using a permeability improving substance.

The poorly permeable active pharmaceutical ingredient to be processed according to this invention can be liquid, semi-solid, solid amorphous or solid crystalline.

The poorly permeable compound to be processed according to this invention can be a pharmaceutically active agent and can be chosen from analgesics, anti-arrhythmic agents, anti-asthma agents, anti-biotic agents, anti-helminthics, anti-inflammatory agents, anti-viral agents, anti-coagulants, anti-depressants, anti-diabetics, anti-epileptics, anti-erectile dysfunction agents, anti-fungal agents, anti-gout agents, anti-hypertensive agents, anti-malarials, anti-migraine agents, anti-muscarinic agents, anti-neoplastic agents, anti-obesity agents, anti-parkinsonian agents, anti-protozoal agents, anti-thyroid agents, anti-tussives, anxiolytics, beta-blockers, hypnotics, immunosuppressants, neuroleptics, cannabinoid receptor agonists and antagonists, cardic inotropic agents, cell adhesion inhibitors, corticosteroids, cytokine receptor activity modulators, diuretics, gastrointestinal agents, histamine H-receptor antagonists, keratolytics, lipid regulating agents, muscle relaxants, nitrates and other anti-anginal agents, non-steroid anti-asthma agents, opioid analgesics, sedatives, sex hormones and stimulants.

An acceptable class of poorly soluble compounds include poorly soluble (3S)-3-[[[1-[2-(2S)-carboxy-4-[[3-(dimethylamino)propyl]methylamino]-4-oxobutyl]cyclopentyl]carbonyl]amino]-2,3,4,5-tetrahydro-2-oxo-1H-1-benzazepine-1-acetic acid, 4-[2-[[[(2S)-1-[(4′-fluoro[1,1′-biphenyl]-4-yl)sulfonyl]-2,3-dihydro-1H-indol-2-yl]carbonyl]amino]ethoxy]benzoic acid, 4-[[[[(2S)-2,3-dihydro-1-[[2′,4′-difluoro[1,1′-biphenyl]-4-yl]sulfonyl]-1H-indol-2-yl]carbonyl]amino]methyl]benzeneacetic acid and the like.

A water soluble carrier according to the present invention should be pharmaceutically acceptable. The pharmaceutically acceptable carrier can be chosen from
alkylcelluloses, such as methylcellulose;
hydroxyalkylcelluloses, such as hydroxymethylcellulose, hydroxyethylcellulose, hydroxypropylcellulose and hydroxybutylcellulose;
hydroxyalkyl alkylcelluloses, such as hydroxyethyl methylcellulose and hydroxypropyl-methylcellulose;
carboxyalkylcelluloses, such as carboxymethylcellulose;
alkali metal salts of carboxyalkylcelluloses, such as sodium carboxymethylcellulose;
carboxyalkylalkylcelluloses, such as carboxymethylethylcellulose;
carboxyalkylcellulose esters;
starches;
pectines, such as sodium carboxymethylamylopectine;
chitin derivates, such as chitosan;
polysaccharides, such as alginic acid, alkali metal and ammonium salts thereof,
carrageenans, galactomannans, tragacanth, agar-agar, gummi arabicum, guar gummi and xanthan gummi;
polyacrylic acids and the salts thereof;
polymethacrylic acids and the salts thereof, methacrylate copolymers;
polyvinylalcohol;
polyvinylpyrrolidone, copolymers of polyvinylpyrrolidone with vinyl acetate; and
polyalkylene oxides, such as polyethylene oxide and polypropylene oxide and copolymers of ethylene oxide and propylene oxide.
   The water soluble carrier is normally soluble in water at room temperature but sometimes forms a dispersion when contacted with water at higher temperature and dissolves totally when the temperature is decreased to room temperature. Therefore when referring to a mixture containing a water soluble carrier this mixture can be a dispersion or a solution.

Non-enumerated polymers which are pharmaceutically acceptable and have appropriate physico-chemical properties as defined hereinbefore are equally suited as a carrier in the present invention for pharmaceutical compositions.

Acceptable water-soluble polymers include hydroxypropylmethylcelluloses (HPMC). Said HPMC contains sufficient hydroxypropyl and methoxy groups to render it water-soluble. HPM's having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxy-propyl molar substitution refers to the average number of moles of propylene oxide which have reacted with each anhydroglucose unit of the cellulose molecule. Hydroxypropyl methylcellulose is the United States Adopted Name for hypromellose.

The composition according to the present invention may include one or more other auxiliary materials. In the case of a pharmaceutical composition these auxiliary materials should be pharmaceutically acceptable additives such as flavoring agents, colorants, binders, fillers, filler-binders, lubricants, disintegration aids and/or other pharmaceutically acceptable additives. In the framework of the present invention auxiliary materials does not include a significant amount of volatile organic solvents. Volatile organic solvents are defined as organic solvents having a vapor pressure higher than 0.50 mm Hg at 25° C. A significant amount is an amount higher than 1% w/w. The auxiliary materials can contain less than 0.5% volatile organic solvents, less than 0.3%, less than 0.1%, as well as less than 0.01% w/w.

### Preparation

The preparation of a matrix composition according to the present invention involves the preparation of an aqueous solution, aqueous micellar solution, an aqueous emulsion, aqueous microemulsion or aqueous nanoemulsion of a permeation enhancing substance followed by a drying step to embed the micelles, emulsion, microemulsion or nanoemulsion in a water-soluble matrix of a carrier, such as a pharmaceutically acceptable carrier.

In a first aspect, the invention relates to a process of preparing a solid thermostable pharmaceutical composition as described above, comprising
a) dissolving or dispersing at least one permeability improving substance in water to form a mixture;
b) dissolving water soluble matrix forming material in the mixture obtained in a) or adding a solution of water soluble matrix forming material in water to the mixture obtained in a);
c) optionally adding one or more additional auxiliary materials to the mixture obtained in a) or b); and
d) drying the mixture obtained in b or c);

In a further aspect, the invention relates to a process of preparing a solid thermostable pharmaceutical composition as described above, comprising the following steps:
a) dissolving or dispersing water soluble matrix forming material in water to form a solution;
b) dissolving or dispersing at least one permeability improving substance in the solution obtained in a) or adding a solution or dispersion of the at least one permeability improving substance in water to the solution obtained in a);
c) optionally adding one or more additional auxiliary materials to the mixture obtained in a) or b); and
d) drying the mixture obtained in b or c)

The thermostable composition obtained via the methods indicated above can be further processed to a final dosage in the form of a mixture with the active pharmaceutical ingredient which may separately be formulated into a powder, granules, pellets or microspheres.

In a further aspect, the invention relates to a process of preparing a solid pharmaceutical composition comprising a water soluble active pharmaceutical ingredient having a bad permeability as described above, comprising the following steps:
a) dissolving or dispersing water soluble matrix forming material in water to form a mixture;
b) dissolving or dispersing the at least one permeability improving substance in the solution obtained in a) or adding a solution or dispersion of the at least one permeability improving substance in water to the mixture obtained in a);
c) optionally adding one or more additional auxiliary materials to the mixture obtained in a or b); and
d) spraying the mixture obtained under b) or c) in the form of a thermostable coating layer onto drug particles of the poorly permeable API, or onto tablets, pellets, granules or capsules containing the poorly permeable API.

In a further aspect, the invention relates to a process of preparing a solid pharmaceutical composition comprising a water soluble active pharmaceutical ingredient having a bad permeability as described above, comprising the following steps:
a) dissolving or dispersing at least one permeability improving substance in water to form a mixture;
b) dissolving water soluble matrix forming material in the mixture obtained in a) or adding a solution of water soluble matrix forming material in water to the mixture obtained in a);
c) optionally adding one or more additional auxiliary materials to the mixture obtained in a) or b); and
d) spraying the mixture obtained under b) or c) in the form of a thermostable coating layer onto drug particles of the poorly permeable API, or onto tablets, pellets, granules or capsules containing the poorly permeable API.

Processes comparable to the processes described above can also be used to prepare a product comprising an API, a permeation enhancer and a water soluble carrier in a single drying step. Therefore, in a further aspect, the invention also relates to a process of preparing a pharmaceutical composition comprising a water soluble active pharmaceutical ingredient having a bad permeability, said process comprising the steps for preparing a thermostable pharmaceutical composition as described above, wherein said active pharmaceutical ingredient is separately dissolved and mixed, before the total mixture is dried, with:
i) the solution of the water soluble matrix forming material in water; or
ii) the solution or dispersion of the at least one permeability improving substance in water; or
iii) one or more additional auxiliary materials;
   or wherein said active pharmaceutical ingredient is dissolved in the solution i) or mixture ii) defined above, or in the solution of the one or more additional auxiliary materials; and wherein the aqueous mixture obtained is dried.

The final formulation formed by applying one of the processes described above is physically stable and remains stable when heated above the melting temperature of the main permeation improving substance and even when the ratio between the matrix forming material and the permeability improving substance is very low, such as lower than 50%, even lower than 30%, even lower than 20%, or even when 10%.

The following examples are only intended to further illustrate the invention, in more detail, and therefore these examples provided herein are not deemed to restrict the scope of the invention in any way

### EXAMPLE 1

### Preparation of a P-gp Inhibiting Formulation System (Permeability Improving Substance is Labrasol (polyglycolyzed glycerides))

Formulation per capsule:

| | |
|---|---|
| API: Poorly permeable active pharmaceutical ingredient: | 150.0 mg |
| (3S)-3-[[[1-[2-(2S)-carboxy-4-[[3- | |
| (dimethylamino)propyl]methylamino]-4- | |
| oxobutyl]cyclopentyl]carbonyl]amino]-2,3,4,5-tetrahydro- | |
| 2-oxo-1H-1-benzazepine-1-acetic acid | |

Further Ingredients:

| | |
|---|---|
| Monobasic sodium phosphate: | 17.0 mg |
| Disodium hydrogen phosphate: | 61.5 mg |
| Carbopol ® 971P (Polymer of 2-propenoic acid): | 12.5 mg |
| Sodium hydroxide: | 6.0 mg |
| Labrasol ® (polyglycolyzed glycerides): | 50.0 mg |
| HPMC E6: | 50.0 mg |
| Water | |

Monobasic sodium phosphate and disodium hydrogen phosphate were dissolved in water to obtain a pH of 7.5. Carbopol® 971 P was added to the buffer solution and dissolved. (3S)-3-[[[1-[2-(2S)-carboxy-4-[[3-(dimethylamino)propyl]methylamino]-4-oxo-butyl]cyclopentyl]carbonyl]amino]-2,3,4,5-tetrahydro-2-oxo-1H-1-benzazepine-1-acetic acid was dissolved in the buffer/Carbopol® 971 P solution while adding sodium hydroxide 2M solution, keeping the pH above 6.0. This final solution was freeze dried (T=−80° C., p=0.002 mbar) for 60 hours. The powder was compressed into a plug, using a die with a diameter of 5.5 mm at a pressure of 0.8 ton (8000 psi) for 1 second. The plug was removed and grinded into small granules. A capsule size 2 was filled with the granules and closed.

A solution of 10% m/m of HPMC E6 was prepared by heating water to a temperature of approximately 65° C. HPMC E6 was added to the heated water and stirred until a homogeneous suspension was formed. The suspension was left cooling, and resulted in a clear solution of HPMC E6 (10% m/m) in water.

Labrasol® was dispersed in the aqueous HPMC E6 solution and spray dried (INLET temperature=145° C., OUTLET temperature=88° C.) to obtain a powder where Labrasol® is thermostable embedded in a HPMC E6 matrix. A capsule size 00 was filled with a size 2 capsule containing the granulate containing (3S)-3-[[[1-[2-(2S)-carboxy-4-[[3-(dimethylamino)propyl]methylamino]-4-oxobutyl]cyclopentyl]carbonyl]amino]-2,3,4,5-tetrahydro-2-oxo-1H-1-benzazepine-1-acetic acid and Carbopol® 971P, furthermore the powder containing the thermostable embedded Labrasol® was added to this external capsule.

### EXAMPLE 2

### Preparation of a P-gp Inhibiting Formulation System (Permeability Improving Substance is Tween 80 (Polyoxyethylene (20) sorbitan monooleate))

Formulation per batch:

| Ingredients: | | |
|---|---|---|
| Tween ® 80: | 45 | g |
| HPMC E6: | 45 | g |
| Water: | 405 | ml |

Tween® 80 was dissolved in water while heating to a temperature of approximately 65° C. HPMC E6 was added to the heated solution and stirred until a homogeneous dispersion was formed.

The dispersion was left cooling and spray dried (INLET temperature=145° C., OUTLET temperature=90° C.) to obtain a powder where Tween® 80 is thermostable embedded in a HPMC E6 matrix.

The obtained powder can be mixed with regular excipients and at least one poorly permeable active pharmaceutical ingredient to obtain a final oral dosage form with P-gp inhibiting capacities.

### EXAMPLE 3

### Preparation of a P-gp Inhibiting Formulation System (Permeability Improving Substance is TPGS (d-alpha-tocopheryl polyethylene glycol 1000 succinate))

Formulation per tablet:

| | |
|---|---|
| API: Poorly permeable active pharmaceutical ingredient: | 300.0 mg |
| (3S)-3-[[[1-[2-(2S)-carboxy-4-[[3- | |
| (dimethylamino)propyl]methylamino]-4- | |
| oxobutyl]cyclopentyl]carbonyl]amino]-2,3,4,5- | |
| tetrahydro-2-oxo-1H-1-benzazepine-1-acetic acid | |

Further Ingredients:

| | |
|---|---|
| TPGS: | 12.5 mg |
| HPMC E6: | 12.5 mg |
| Microcrystalline cellulose: | 102.6 mg |
| Primojel ® (sodium starch glycolate): | 102.6 mg |
| Aerosil ® 200V (amorphous anhydrous colloidal | 2.6 mg |
| silicon dioxide): | |
| PRUV ® (sodium stearyl fumarate): | 5.1 mg |
| Water | |

TPGS was dispersed in water while heating to a temperature of approximately 65° C. HPMC E6 was added to the heated solution and stirred until a homogeneous suspension was formed. The suspension was left cooling, and a homogeneous dispersion was obtained.

(3S)-3-[[[1-[2-(2S)-carboxy-4-[[3-(dimethylamino)propyl]methylamino]-4-oxobutyl]-cyclopentyl]carbonyl]amino]-2,3,4,5-tetrahydro-2-oxo-1H-1-benzazepine-1-acetic acid was mixed together with microcrystalline cellulose, Primojel®, Aerosil® and PRUV®. A tablet was pressed using the powder mixture, containing (3S)-3-[[[1-[2-(2S)-carboxy-4-[[3-(dimethylamino)propyl]methylamino]-4-oxobutyl]cyclopentyl]-carbonyl]amino]-2,3,4,5-tetrahydro-2-oxo-1H-1-benzazepine-1-acetic acid.

The obtained dispersion of TPGS and HPMC E6 was heated to approximately 60° C. and sprayed in the form of a coating layer onto the core tablets containing (3S)-3-[[[1-[2-(2S)-carboxy-4-[[3-(dimethylamino)propyl]methylamino]-4-oxobutyl]cyclopentyl]-carbonyl]amino]-2,3,4,5-tetrahydro-2-oxo-1H-1-benzazepine-1-acetic acid, resulting in a coating where TPGS is thermostable and embedded in a HPMC E6 matrix.

### EXAMPLE 4

### Preparation of a P-gp Inhibiting Formulation System (Permeability Improving Substance is Solutol® HS 15 (polyoxyethylene esters of 12-hydroxystearic acid))

Formulation per batch:

| Ingredients: | | |
|---|---|---|
| Solutol ® HS 15: | 45 | g |
| HPMC E6: | 45 | g |
| Water: | 810 | ml |

A solution of 10% m/m of Solutol® HS 15 was prepared by dissolving the Solutol® HS 15 in water.

A solution of 10% m/m of HPMC E6 was prepared by heating water to a temperature of approximately 65° C. HPMC E6 was added to the heated water and stirred until a homogeneous suspension was formed. The suspension was left cooling, and resulted in a clear solution of HPMC E6 (10% m/m) in water.

Both solutions were mixed together and spray dried (INLET temperature=145° C., OUTLET temperature=90° C.) to obtain a powder where Solutol® HS 15 is thermostable embedded in a HPMC E6 matrix.

The obtained powder was mixed with regular excipients and at least one poorly permeable active pharmaceutical ingredient to obtain a final oral dosage form with P-gp inhibiting capacities.

### EXAMPLE 5

### Preparation of a P-gp Inhibiting Formulation System (Permeability Improving Substance is Gelucire® 44/14 (PEG-32 glyceryl laurate))

Formulation per batch:

| Ingredients: | | |
|---|---|---|
| Gelucire ® 44/14: | 45 | g |
| HPMC E6: | 45 | g |
| Water: | 405 | ml |

A solution of 10% m/m of HPMC E6 was prepared by heating water to a temperature of approximately 65° C. HPMC E6 was added to the heated water and stirred until a homogeneous suspension was formed. The suspension was left cooling, and resulted in a clear solution of HPMC E6 (10% m/m) in water.

The obtained HPMC E6 solution was heated up to approximately 65° C. and Gelucire® 44/14 was dispersed in this aqueous solution. The dispersion was left cooling and spray dried (INLET temperature=145° C., OUTLET temperature=90° C.) to obtain a powder where Gelucire® 44/14 was thermostable and embedded in a HPMC E6 matrix.

The obtained powder was mixed with regular excipients and at least one poorly permeable active pharmaceutical ingredient to obtain a final oral dosage form with P-gp inhibiting capacities.

### EXAMPLE 6

### Preparation of a P-gp Inhibiting Formulation System (Permeability Improving Substance is TPGS (d-alpha-tocopheryl polyethylene glycol 1000 succinate))

Formulation per tablet:

| | |
|---|---|
| API: Poorly permeable active pharmaceutical ingredient: | 300.0 mg |
| (3S)-3-[[[1-[2-(2S)-carboxy-4-[[3- | |
| (dimethylamino)propyl]methylamino]-4- | |
| oxobutyl]cyclopentyl]carbonyl]amino]-2,3,4,5- | |
| tetrahydro-2-oxo-1H-1-benzazepine-1-acetic acid | |

Further Ingredients:

| | |
|---|---|
| TGPS: | 12.5 mg |
| HPMC E6: | 12.5 mg |
| Microcrystalline cellulose: | 102.6 mg |
| Primojel ® (sodium starch glycolate): | 102.6 mg |
| Aerosil ® 200V (amorphous anhydrous | 2.6 mg |
| colloidal silicon dioxide): | |
| PRUV ® (sodium stearyl fumarate): | 5.1 mg |
| Water | |

A solution of 10% m/m of HPMC E6 was prepared by heating water to a temperature of approximately 65° C. HPMC E6 was added to the heated water and stirred until a homogeneous suspension was formed. The suspension was left cooling, and resulted in a clear solution of HPMC E6 (10% m/m) in water.

The obtained HPMC E6 solution was heated up to approximately 65° C. and TPGS was dispersed in this aqueous solution. The dispersion was left cooling.

(3S)-3-[[[1-[2-(2S)-carboxy-4-[[3-(dimethylamino)propyl]methylamino]-4-oxobutyl]-cyclopentyl]carbonyl]amino]-2,3,4,5-tetrahydro-2-oxo-1H-1-benzazepine-1-acetic acid was mixed together with microcrystalline cellulose, Primojel®, Aerosil® and PRUV®. A tablet was pressed using the powder mixture, containing (3S)-3-[[[1-[2-(2S)-carboxy-4-[[3-(dimethylamino)propyl]methylamino]-4-oxobutyl]cyclopentyl]carbonyl]amino]-2,3,4,5-tetrahydro-2-oxo-1H-1-benzazepine-1-acetic acid.

The obtained dispersion of TPGS and HPMC E6 was heated up to approximately 60° C. and was sprayed to form a coating layer onto the core tablets containing (3S)-3-[[[1-[2-(2S)-carboxy-4-[[3-(dimethylamino)propyl]methylamino]-4-oxobutyl]cyclopentyl]-carbo-nyl]amino]-2,3,4,5-tetrahydro-2-oxo-1H-1-benzazepine-1-acetic acid, resulting in a coating where TPGS is thermostable embedded in a HPMC E6 matrix.

### EXAMPLE 7

### Preparation of a P-gp Inhibiting Formulation System (Permeability Improving Substance is Labrasol® (polyglycolyzed glycerides))

Formulation per tablet:

| | |
|---|---|
| API: Poorly permeable active pharmaceutical ingredient: | 300.0 mg |
| (3S)-3-[[[1-[2-(2S)-carboxy-4-[[3- | |
| (dimethylamino)propyl]methylamino]-4- | |
| oxobutyl]cyclopentyl]carbonyl]amino]-2,3,4,5- | |
| tetrahydro-2-oxo-1H-1-benzazepine-1-acetic acid | |

Further Ingredients:

| | |
|---|---|
| Labrasol ®: | 12.5 mg |
| HPMC E6: | 12.5 mg |
| Microcrystalline cellulose: | 102.6 mg |
| Primojel ® (sodium starch glycolate): | 102.6 mg |
| Aerosil ® 200V (amorphous anhydrous | 2.6 mg |
| colloidal silicon dioxide): | |
| PRUV ® (sodium stearyl fumarate): | 5.1 mg |
| Water | |

A dispersion of 10% m/m of Labrasol® was prepared by dispersing the Labrasol® in water. A solution of 10% m/m of HPMC E6 was prepared by heating water to a temperature of approximately 65° C. HPMC E6 was added to the heated water and stirred until a homogeneous suspension was formed. The suspension was left cooling, and resulted in a clear solution of HPMC E6 (10% m/m) in water. Both solutions were mixed together.

(3S)-3-[[[1-[2-(2S)-carboxy-4-[[3-(dimethylamino)propyl]methylamino]-4-oxobutyl]-cyclopentyl]carbonyl]amino]-2,3,4,5-tetrahydro-2-oxo-1H-1-benzazepine-1-acetic acid was mixed together with microcrystalline cellulose, Primojel®, Aerosil® and PRUV®. A tablet was pressed using the powder mixture, containing (3S)-3-[[[1-[2-(2S)-carboxy-4-[[3-(dimethylamino)propyl]methylamino]-4-oxobutyl]cyclopentyl]carbonyl]amino]-2,3,4,5-tetrahydro-2-oxo-1H-1-benzazepine-1-acetic acid.

The obtained dispersion of Labrasol® and HPMC E6 was sprayed to form a coating layer onto the core tablets containing (3S)-3-[[[1-[2-(2S)-carboxy-4-[[3-(dimethyl-amino)propyl]methylamino]-4-oxobutyl]cyclopentyl]carbonyl]amino]-2,3,4,5-tetrahydro-2-oxo-1H-1-benzazepine-1-acetic acid, which resulted in a coating where Labrasol® was thermostably embedded in a HPMC E6 matrix.

### EXAMPLE 8

### Preparation of a Formulation Where the Active Pharmaceutical Ingredient is Spray-Dried Together with the Permeability Improving Substance

API: Poorly permeable active pharmaceutical ingredient: (3S)-3-[[[1-[2-(2S)-carboxy-4-[[3-(dimethylamino)propyl]methylamino]-4-oxobutyl]cyclopentyl]carbonyl]-amino]-2,3,4,5-tetrahydro-2-oxo-1H-1-benzazepine-1-acetic acid

Step 1) Preparation of a Mixture Containing Various Permeability Improving Substances.

Approximately 50 g of sodium caprylate was weighed into a glass plasma flacon (250 ml) and dissolved in approximately 150 g of caprylic acid (Part A). Approximately 90 g of Capmul MCM C8 was weighed into a glass plasma flacon (500 ml) and approximately 180 g of Captex 8000 was added and homogenised (Part B). Approximately 200 g of Solution A was added to Solution B and homogenised. The resulting mixture contained therefore various permeability improving substances in the form of a microemulsion system.

Step 2) Preparation of a Thermostable Powder Formulation Containing an Active Pharmaceutical Ingredient and a Various Permeability Improving Substances

A 2.2% m/m HPMC E50LV solution was made up by dissolving 100 g of HPMC E50LV in 1487 g of purified water at approximately 70° C. Sodium hydrogen phosphate.2H₂O (7.6832 g), sodium dihydrogen phosphate. H₂O (1.0513 g) and sodium hydroxide (0.5980 g) were added to this solution. 100 g of API was dissolved in this solution and an additional amount of purified water (1327 g) at approximately 68° C. was added and cooled to room temperature under continuous stirring. The spraying solution was prepared by adding 100 g of the mixture containing various permeability improving substances (prepared in step 1) to this HPMC E50LV solution and homogenised.

This solution was sprayed, using a Büchi B-191 mini spray-dryer:

| | |
|---|---|
| Tᵢₙ: | 140° C. |
| Tₒᵤₜ: | 80° C. |
| Aspirator: | 90% |
| Flow: | 600 L/h |
| Nozzle: | 0.7 mm |
| Application rate: | 25% |
| ΔP_{filter}: | ±−20 mbar |

The resulting thermostable powder contained an active pharmaceutical ingredient and various permeability improving substances.

Step 3) Compression to Tablets

The powder produced in step 2 was further processed to tablets. Therefore a blend was made by weighing approximately 465 mg of the powder produced in step 2 together with 143 mg microcrystalline cellulose PH200, 143 mg Primojel and mixed. Tablets were compressed using a hydraulic press:

| | |
|---|---|
| P: | 200 bar |
| t: | ~2 s |
| Ø: | 19 × 8.4 mm oblong, double concave. |
| Mass: | 750 mg |

1. A thermostable solid composition comprising at least one permeability improving substance embedded in a water soluble matrix, wherein the sum of the amount of said permeability improving substance or mixture of permeability improving substances and said water soluble matrix is at least 80% w/w of the total dry material in the composition, with the proviso that said thermostable solid composition does not contain an active pharmaceutical ingredient.

2. The thermostable solid composition according to claim 1, wherein the at least one permeability improving substance is chosen from d-alpha tocopheryl polyethylene glycol 1,000 succinate (Vit E TPGS), PEG-32 glyceryl laurate, caprylic/capric acid triglyceride, glyceryl monocaprylate, glyceryl mono-di-caprylate, polyethoxylated castor oil, polyglycolyzed glycerides and polyoxyethylene esters of 12-hydroxystearic acid, medium chain triglycerides, caprylocaproyl macrogol-8 glycerides, polyoxyethylene-20 sorbitanmonooleate, macrogol-15 hydroxystearate, propylene glycol-monocaprylate, propylene gycol-caprylcaprate, and propylene glycol-monolaurate.

3. The thermostable solid composition according to claim 2, wherein the at least one permeability improving substance is Labrasol®, Solutol® HS 15, Capmul® MCM C8, Captex® 8000, Vitamin E TPGS, Gelucire® 44/14, Cremophor® EL, Tween® 80, Miglyol® 812, Capryol® 90, Capryol® PGMC, Labrafac® PG, Lauroglycol® 90, or Lauroglycol® FCC.

4. A pharmaceutical composition comprising at least two phases, wherein the first phase a) comprises an active pharmaceutical ingredient formulated into a powder, granules, pellets, microspheres or a tablet, and the second phase b) comprises a thermostable solid composition according to any of claims 1-3, wherein said active pharmaceutical ingredient is a poorly permeable water soluble substance (BCS III compound).

5. The pharmaceutical composition according to claim 4, wherein the at least two phases are mixed and packaged in a capsule.

6. The pharmaceutical composition according to claim 4, wherein the second phase is applied as a coating on the first phase.

7. A pharmaceutical composition according to any of claims 4-6, wherein the active pharmaceutical ingredient has a bad permeability.

8. A process of preparing a thermostable solid composition comprising at least one permeability improving substance embedded in a water soluble matrix, said process comprising:
a) dissolving or dispersing at least one permeability improving substance in water to form a mixture;
b) dissolving water soluble matrix forming material in the mixture obtained in a) or adding a solution of water soluble matrix forming material in water to the mixture obtained in a);
c) optionally adding one or more auxiliary materials to the mixture obtained in a) or b); and
d) drying the mixture obtained in b) or c);
wherein the sum of the amount of said permeability improving substance or mixture of permeability improving substances and said water soluble matrix is at least 80% w/w of the total dry material of the composition.

9. A process of preparing a thermostable solid composition comprising at least one permeability improving substance embedded in a water soluble matrix, said process comprising:
a) dissolving or dispersing water soluble matrix forming material in water to form a mixture;
b) dissolving or dispersing at least one permeability improving substance in the mixture obtained in a), or adding a solution or dispersion of the at least one permeability improving substance in water to the mixture obtained in a);
c) optionally adding one or more auxiliary materials to the mixture obtained in a or b); and
d) drying the mixture obtained in b or c);
wherein the sum of the amount of said permeability improving substance or mixture of permeability improving substances and said water soluble matrix is at least 80% w/w of the total dry material of the composition.

10. A process of preparing a pharmaceutical composition comprising a water soluble active pharmaceutical ingredient having a bad permeability, said process comprising the steps of claims 8 to 9, wherein said active pharmaceutical ingredient is separately dissolved and mixed, before the total mixture is dried, with:
i) the solution of the water soluble matrix forming material in water; or
ii) the solution or dispersion of the at least one permeability improving substance in water; or
iii) one or more auxiliary materials;
or wherein said active pharmaceutical ingredient is dissolved in the solution i) or mixture ii) defined above, or in the solution of the one or more additional auxiliary materials; and wherein the aqueous mixture obtained is dried; and wherein the sum of the water soluble matrix forming material and the at least one permeability improving substance is at least 80% w/w of the dry material, excluding the active pharmaceutical ingredient.

11. The process according to any of claims 8-10, wherein the drying is chosen from spray-drying, spray-coating, spray-layering, spray-granulation, freeze-drying, and spray freeze-drying.

12. A process of preparing a pharmaceutical composition according to claim 4, comprising the mixing of an active pharmaceutical ingredient formulated into a powder, granules, pellets or microspheres with a thermostable solid composition according to any of claims 1-3.

13. A process of preparing a pharmaceutical composition according to claim 4, comprising the spraying of an aqueous solution of a thermostable solid composition according to claims 1-3 onto an active pharmaceutical ingredient formulated as granules, pellets, microspheres or as a tablet.

14. A method of improving the bioavailability of an active pharmaceutical ingredient, comprising
a) mixing a thermostable solid composition according to any of claims 1-3 with an active pharmaceutical ingredient formulated as a powder, granules, a pellets or microspheres; or
b) spraying a thermostable solid composition according to any of claims 1-3 on an active pharmaceutical ingredient formulated as granules, pellets, microspheres or as a tablet.

15. A product comprising a water soluble active pharmaceutical ingredient having a bad permeability, said product prepared according to the method described in any of claims 7-12, wherein said water soluble active pharmaceutical ingredient is chosen from (3S)-3-[[[1-[2-(2S)-carboxy-4-[[3-(dimethylamino)propyl]-methylamino]-4-oxobutyl]cyclopentyl]carbonyl]amino]-2,3,4,5-tetrahydro-2-oxo-1H-1-benzazepine-1-acetic acid and 4-[2-[[[(2S)-1-[(4′-fluoro[1,1′-biphenyl]-4-yl)sulfonyl]-2,3-dihydro-1H-indol-2-yl]carbonyl]amino]ethoxy]benzoic acid and 4-[[[[(2S)-2,3-dihydro-1-[[2′,4′-difluoro[1,1′-biphenyl]-4-yl]sulfonyl]-1H-indol-2-yl]carbonyl]amino]methyl]-benzeneacetic acid.

16. A pharmaceutical composition according to any of claims 4-7, wherein said active pharmaceutical ingredient is chosen from (3S)-3-[[[1-[2-(2S)-carboxy-4-[[3-(dimethylamino)propyl]methylamino]-4-oxobutyl]cyclopentyl]carbonyl]amino]-2,3,4,5-tetrahydro-2-oxo-1H-1-benzazepine-1-acetic acid and 4-[2-[[[(2S)-1-[(4′-fluoro[1,1′-biphenyl]-4-yl)sulfonyl]-2,3-dihydro-1H-indol-2-yl]carbonyl]amino]ethoxy]benzoic acid and 4-[[[[(2S)-2,3-dihydro-1-[[2′,4′-difluoro[1,1′-biphenyl]-4-yl]sulfonyl]-1H-indol-2-yl]carbonyl]amino]methyl]benzeneacetic acid.
